(19) United States
(12) Patent Application Publication
(10) Pub. No.: US 2005/0260142 A1
(43) Pub. Date: Nov. 24, 2005

(54) Method for tooth whitening, in particular a system for tooth whitening using a phase transfer catalyst (PTC)

(76) Inventors: Graham K. Philp JR., Naples, FL (US); Damon Brown, Boston, MA (US)

Correspondence Address: SHLESINGER, ARKWRIGHT & GARVEY LLP, 1420 KING STREET, SUITE 600, ALEXANDRIA, VA, 22314, US

(21) Appl. No.: 11/116,429

(22) Filed: Apr. 28, 2005

Related U.S. Application Data
(60) Provisional application No. 60/565,841, filed on Apr. 28, 2004.

(51) Int. Cl. A61K 7/20

(52) U.S. Cl. 424/53

(57) ABSTRACT

Tooth whitening composition includes an oxidizing compound and phase transfer catalyst. The phase transfer catalyst may be administrated in the same or in a different composition. The phase transfer catalyst may be incorporated into the tooth whitening composition itself, or it may be included in a composition formulated to contact the enamel surface of a patient prior to the treatment of a treated tooth surface with the oxidizing compound, or both.

### CROSS-REFERENCE TO RELATED APPLICATION

This application claims the priority of application No. 60/565,841, filed Apr. 28, 2004, which is incorporated herein by reference.

### FIELD OF THE INVENTION

The present invention relates to a tooth whitening composition and methods for tooth whitening using phase transfer catalysis (PTC).

### BACKGROUND OF THE INVENTION

Tooth structures, which become discolored with age, blood, amalgam restoration, antibiotics, substances in food, beverages, and tobacco are enamel, dentin, and the acquired pellicle.

Tooth enamel is predominately formed from an inorganic substance, hydroxyapatite crystals along with approximately 5% organic materials, predominately collagen.

The dentin is composed of about 20% protein including collagen, the balance including organic materials and hydroxyapatite crystals.

The acquired pellicle is a proteinaceous layer on the surface of the tooth enamel, which can be removed by mechanical expedients such as intensive tooth cleaning.

Tooth bleaching to achieve lighter (i.e., whiter) teeth, is considered to be very desirable in today's cosmetically—orientated society. Tooth bleaching has been generally accomplished by gels, pastes, or liquids, which contains an oxidizing agent such as hydrogen peroxide that can form oxygen free radicals (nascent oxygen species), which will attack the chromogen (stain) molecules, making them colorless and/or water soluble or both resulting in the teeth being lighter and brighter appearing.

The most commonly used oxidizing agent is hydrogen peroxide, which is obtained in a liquid form, and which is mixed with an anhydrous carrier containing glycerin and/or propylene glycol and/or polyethylene glycol. Also commonly used is carbamide peroxide (urea hydrogen peroxide) which can be dissolved in ethanol and which upon contact with water dissociates into urea and hydrogen peroxide. The hydrogen peroxide in the presence of water then dissociates into water and an oxygen free radical. These nascent oxygen species are highly reactive and react with the stain molecules making them water soluble and/or transparent or both.

The greatest oxidizing activity is required at the tooth surface and within the first few millimeters of the enamel or dentin. There have been several delivery systems to deliver the oxidizing agent to the surfaces of the teeth. A common approach is to have a dental professional construct a custom-made tray from an impression resulting in a cast of the patient's teeth. The oxidizing gel is dispensed into the tray by the patient and the tray is worn over the teeth intermittently for a period of two weeks to several months depending on the severity of the staining. This approach can cause sensitivity in over 50% of the patients and it can be uncomfortable and awkward for the patients to wear the tooth bleaching trays. Tooth sensitivity is believed to result from movement of fluid through the dentinal tubules, which is sensed by nerve endings within the tooth. The carriers glycerine, propylene glycol and polyethylene glycol can contribute to the tooth sensitivity often experienced by wearing a bleaching tray.

Another approach is to incorporate the oxidizing agent into a strip and have the patient wear the strip intermittently over a period of two weeks. This approach also has the disadvantage of being awkward for the patients to wear, and many patients wearing the strips also experience tooth sensitivity. Further, the strips have an inherent problem of contacting the teeth only on the facial surfaces and most of the staining occurs in between the teeth in the interproximal areas, where it can be difficult to place the strip, having it in contact with the tooth.

Another recent approach is simply to paint the oxidizing agent, carbamide peroxide or hydrogen peroxide, directly on the tooth. In products containing glycerin and/or propylene glycol or polyethylene glycol, tooth sensitivity can be experienced. Further, these products are gels, and the gels can easily be removed by the lips and tongue decreasing their effectiveness.

One approach dissolves the carbamide peroxide in alcohol with a resin, and when the alcohol is allowed to evaporate away, the resin forms a film or precipate, which is left on the surface of the teeth. When saliva contacts the teeth, the peroxide is released in a relatively strong concentration for a period of up to 20 minutes. This product contains no glycerin or propylene glycol or polyethylene glycol, and tooth sensitivity is virtually non-existent.

These products are easy and simple to use and good results have been obtained; however, they take approximately two weeks of use several times a day to see the desired result.

To overcome the problem of taking excessive time to whiten teeth, manufacturers developed an approach which is used by a dental professional in the dental office, and which can result in whitening the teeth in approximately one hour. These systems generally use oxidizing compositions (hydrogen peroxide up to 35% to 40%) which are applied directly on the teeth in a dental office under the supervision of a dentist or hygienist. Owing to the high concentration of oxidizing agents contained in these office products, they can be hazardous to the patient if not handled carefully, the patients soft tissues—gingiva, lips and tissues—must be isolated from potential exposure to the strong concentration of the oxidizing agent by the use of a rubber dam or by covering the soft tissues with a polymerizable resin that is shaped to conform to the gingival contours. The isolated oxidizing composition is subsequently subjected to a high intensity light source directed at the teeth on which the oxidizing composition has been provided.

Known patents directed to these various tooth whitening systems include:
U.S. Pat. No. 4,952,143 to Becker et al.;
U.S. Pat. No. 5,032,178 to Cornell; and
U.S. Pat. No. 5,785,527 to Jensen et al.

There is a need for a rapid efficient tooth whitening system, method, and composition which is capable of whitening the teeth with a low concentration of oxidizing agent, so that the patient can use the product at home without harming the tissues or burning the tissues or causing mouth/tooth irritation or tooth sensitivity. The tooth whitening method, system, and composition needed needs to be more rapid and efficient than the prior art.

### OBJECTS OF THE INVENTION

It is an object of the invention to provide a tooth whitening method and system that overcomes the drawbacks of prior art methods, systems and devices.

It is a further object of the invention to provide a tooth whitening system which provides rapid tooth whitening without the use of a high concentration of an oxidizing agent.

It is another object of this invention to provide a tooth whitening system which the consumer can use at home or anywhere, thereby being more cost-effective than in-office teeth whitening regimes.

It is a further object of this invention to have a whitening system in which rapid, effective whitening can occur with multiple delivery systems. Tooth whitening is thus possible by the user directly applying a liquid or gel, using a mouth rinse or simply by chewing a tablet or sucking on a lozenge.

It is a further object of this invention to provide rapid tooth whitening which is more efficient in providing oxygen free radicals to the tooth than with previous tooth whitening systems.

Another object is to provide a tooth whitening composition in which a specific phase transfer catalyst is utilized.

A further object is to provide a delivery system utilizing a phase transfer catalyst in combination with a whitening compound and which is not limited to direct application on patient's teeth to be whitened.

Another object of the invention is to provide a phase transfer catalyst which may be incorporated into a pre-treatment application, such as a mouth rinse, applied to the tooth surface of the tooth to be whitened prior to the application of the whitening compound.

Another object of the invention is to provide a phase transfer catalyst specifically selected for carrying and transporting oxygen free radicals (nascent oxygen species) to the chromogen (stain) on teeth to be treated where the oxygen free radicals are released enabling the oxidation of the stain reactions to occur.

A further object of the invention is the utilization of a phase transfer catalyst which will produce rapid and efficient stain removal at a lower concentration of the oxidizing agent, such as hydrogen peroxide, than had been previously possible in prior art stain removal methods.

Another object of the invention is to provide for the use of a phase transfer catalyst which may be utilized in a liquid state, a solid state, or a semi solid state.

A further object of the invention is to provide a tooth whitening system which includes the use of a solid or semi-solid phase transfer catalyst in conjunction with a whitening compound.

A further object of the invention is to provide a tooth whitening system which includes the use of a liquid phase transfer catalyst in conjunction with a whitening compound.

Another object of the invention is to provide a tooth whitening composition including a tooth whitening compound and a phase transfer catalyst which enhances the transfer of the active whitening agent to the user's teeth to be whitened.

Another object of the invention is to provide a tooth whitening composition of the type described above in which the whitening agent includes a nascent oxygen species along with the production of a high concentration of pure oxygen.

Another object of the invention is to provide a tooth whitening system and method including the use of a pretreatment compound for raising the pH of the mouth prior to the whitening treatments.

A further object of the invention is to provide a tooth whitening system in which a pretreatment compound is provided for the user to use in the mouth to raise the pH of the mouth to between about 7.9-9 prior to the application of the whitening agent.

A further object of the invention is to provide one or both of a phase transfer catalyst and a whitening agent in the form of a solid or semi-solid pill.

A further object of the invention is to provide a tooth whitening system in which a solid or semi-solid pill is used by the user to release a whitening agent (e.g., hydrogen peroxide or sodium perborate) for whitening the teeth.

A still further object of the invention is to provide a pretreatment mouth rinse which includes a phase transfer catalyst for enhancing the whitening efficacy of the whitening agent in the tooth whitening compound used by the user whose teeth is to be whitened.

### SUMMARY OF THE INVENTION

Specifically the desired reaction in the tooth whitening process is the oxidation reaction whereby the free oxygen radical (nascent oxygen species) is carried and transported to the stain (chromogen) molecule where the free oxygen radical is released and allowed to react specifically with the stain (chromogen) molecule in an environment of a high concentration of an oxidizing agent. In the present invention this is specifically accomplished by the use of a phase transfer catalyst. This is beneficial in the rapid efficient stain removal of teeth whitening in that it enables more of the free radical oxygen to react with the stain (chromogen) than previously and prevents many of the anions from reacting with each other and forming oxygen which can easily evaporate on the surface film or dissolve into the water which is produced from the breakdown of hydrogen peroxide. Further, as the phase transfer catalyst is utilizing a lower concentration of oxidizing agent (e.g., hydrogen peroxide) it may be used along with alternative delivery systems.

### DETAILED DESCRIPTION OF THE PREFERRED

These embodiments are set forth to describe and illustrate the invention but are not intended to be self-limiting.

The invention achieves rapid tooth whitening by using a phase transfer catalyst which can be incorporated into multiple delivery systems. A phase transfer catalyst is a substance which transports the anion (free radical) into the organic phase so that the free radical can be released and react with the substrate.

The greatest oxidizing activity is required in the first few millimeters of the enamel and in dentin at the tooth surface. The present invention is more effective at removing teeth stains at lower levels of active oxidizing agents than known systems, thereby resulting in a better whitening system which can be used personally (e.g., at home) directly by the consumer and which results in a more rapid tooth whitening.

The oxygen free radicals which are formed from the breakdown of hydrogen peroxide are highly reactive and will react with each other to produce oxygen O² rapidly. It is the ability and quantity of the free radical oxygen radicals to reach the stain molecules and react with them that determines the efficacy of the whitening process. The quantity of the oxygen free radicals produced is directly related to the percent concentration of the hydrogen peroxide. However, it is the quantity of the oxygen free radicals which actually reach and react with the chromogen (stain) molecule which produces the efficacious tooth whitening.

As is well known: H²O²->H2O+O (oxygen free radicals [oxygen anions])

The oxygen free radicals are highly reactive and react with almost anything including themselves to produce oxygen O². The oxygen molecules which are formed from two oxygen free radicals can be evaporated at the surface of the film which is coating the tooth surface, or the oxygen molecules can dissolve in the water which is produced from the breakdown of hydrogen peroxide. In any event, these oxygen free radicals, which form oxygen molecules, are able to react with the stain molecule to make it more transparent and/or water soluble.

Carbamide peroxide (urea hydrogen peroxide) is hydrogen peroxide bound to urea in a stable solid state. It yields only about one-third as much hydrogen peroxide as hydrogen peroxide in its pure form; e.g., what is typically referred to as hydrogen peroxide (and typically supplied and used in its liquid state). However, carbamide peroxide may be very efficient as a tooth whitening agent because the urea molecule is an anion which resembles the protein (collagen) molecule in the organic substrate of the tooth enamel. The amine of the urea will be naturally attracted to the tooth enamel and carry the hydrogen peroxide with it where the oxygen free radicals will be in proximity of the tooth surface. If a phase transfer catalyst were to be utilized with a whitening compound containing carbamide peroxide, the oxygen free radicals produced could very efficiently be carried directly to the teeth (or into the tooth). Once at the teeth the oxygen free radicals are to be released and directly react with the tooth chromogen (stain) molecule. With the phase transfer catalyst fewer oxygen free radicals would be lost by reacting with each other to form oxygen molecules prior to reaching the teeth to be whitened, and very efficient effective tooth whitening would result.

Given that in the prevent invention a phase transfer catalyst would be utilized it is not theoretically necessary to have the hydrogen peroxide in direct proximity with the tooth surface. The phase transfer catalyst would carry the free oxygen radicals and transport them to the substrate; that is the organic substrate of the tooth—where the stain (chromogen) exists. The oxygen free radicals would then be released where they would react with the stain making the stain transparent, water-soluble, or both.

By using a pretreatment mouth rinse with a disinfectant, the peroxidase enzymes would be temporarily eliminated and a phase transfer catalyst would carry the oxygen free radicals.

Therefore, the oxygen free radicals catalyst, and therefore the delivery system, would not be limited to directly applying the hydrogen peroxide or other oxidizing agent. The oxidizing agent could or sucked on (e.g., by the patient sucking on a solid, hard or semi-solid lozenge-type vehicle e.g., a lozenge or similar oxidizing agent delivery vehicle) to obtain the desired whitening result. Further, since the oxygen free radicals would be specifically carried and transported to where the oxygen free radicals are needed to react very efficient whitening would result. That efficient tooth whitening would give very rapid tooth whitening.

It must be understood that the delivery system using a phase transfer catalyst is not limited to use of a wafer or tablet or lozenge-like delivery system; rather, many alternative delivery systems could be employed including direct application on the teeth to be whitened.

In a further embodiment of the invention the phase transfer catalyst may be utilized in a substance or applied prior to the application of the whitening formula. A system would be devised whereby the tooth would be pre-treated or coated with the phase transfer catalyst prior to applying the whitening formula. These embodiments are set forth to illustrate the invention, but must not be construed as limiting.

The phase transfer catalyst acts as a shuttling agent by extracting the anion from the aqueous (or solid) phase into the organic reaction phase (or interfaced region) where the anions can freely react with the organic reactant already located in the organic phase. As one can readily see, phase transfer catalysts have tremendous applications in tooth whitening processes.

### EXAMPLE 1

(liquid phase transfer catalyst+whitening compound)

| | PREFERRED | |
|---|---|---|
| COMPOUND | % BY WEIGHT | RANGE |
| NP-9, ethoxylated | 7.5% | 0.0001-75.00% |
| nonyphenol, 9 molar EO. | | |
| Alcohol | 80% | 0.0001-90.00% |
| Urea (Carbamide) | 12% | 0.0001-25.00% |
| Peroxide | | |
| Hydroxypropylcellulose | 50% | 0.0001-5.00% |

The inventive tooth whitening, system, and method likewise includes a solid/semi-solid phase transfer catalyst and whitening compound usable in addition to or instead of the liquid form of Example 1 described above, and such may be readily provided in such solid or semi solid form as will be apparent to a person having ordinary skill in the art.

Phase transfer catalysis technology is used in the commercial manufacture of chemicals.

Phase transfer catalysis technology is also used in pollution prevention, pollution treatment, and in the removal or destruction of impurities in waste and streams. The scope of phase transfer catalysis technology is broad, as will be appreciated by considering the range of reactions to which phase transfer catalysis technology is applicable. The patents and publications which refer to phase transfer catalysis on the official U.S. PTO website, http://www.uspto.gov, fall into approximately 30 major reaction categories and are responsible for reactions in a wide range of industries:

| | | |
|---|---|---|
| Monomers | Additives | Surfactants |
| Polymers | Flavorant & Fragrances | Petrochemicals |
| Agricultural Chemicals | Dyes | Rubber |
| Pharmaceuticals | Explosives | |

In a search of the literature on tooth whitening, the use of phase transfer catalysts was not discovered and utilized as has been done in the present invention. Some examples of well-know phase transfer catalyzed reactions include:
Nucleophilic substitution reactions, such as halogenations and cyanations
Alkylations and condensation reactions
Oxidation and reductions
Elimination reactions
Wittig and Wittig-House reactions

The reaction with tooth whitening is an oxidation reaction

The requested search for Phase Transfer Catalyses performed on the U.S.P.T.O. database fell into four (4) basic categories:

1. Ammonium based Phase Transfer Catalysis Reagents: these can be toxic and therefore not applicable to tooth whitening;

2. Phosphonium based Phase Transfer Catalysis Reagents Tributylhexadecylphosphonium bromide;
Tetrabutylphosphonium chloride
Tetrabutylphosphonium bromide

3. Crown esters Phase Transfer Catalysis Reagents
12-Crown-4
1-Aza-15-crown-5
15-crown-5
Dibenzo-18-crown-6
Dibenzo-24-crown-8
18-Crown-6
Dicyclohexamo-18-crown-6
Dicyclohexamo-24-crown 8
This (2-(2-methoxyethoxy) ethyl) amino
4, 7, 13, 16, 21, 24-Hexaova-1,10-diazabicyclo (8, 8, 8) hexacosane; and

4. Phase transfer catalysis based on Polymer Support
Tributylmethylammonium chloride-polymer bound
Hexyltributylphosphonium bromide on polymer support
Tributylmethylphosphonium chloride-polymer bound
Hexamethylphosphoramide-polymer bound
Polyethylene glycol 600 monobenzyl ether polymer bound
Polyethylene glycol 750-monobenzyl monomethyl ether polymer bound.

While this invention has been described as having a preferred design, it is understood that it is capable of further modifications, and uses and/or adaptations of the invention and following in general the principle of the invention and including such departures from the present disclosure as come within the known or customary practice in the art to which the invention pertains, and as may be applied to the central features hereinbefore set forth, and fall within the scope of the invention.

### APPENDIX

### Full Names Below (Alphabetical)

Quaternary Phosphonium Salts

Benzyl Triphenyl Phosphonium Bromide
Benzyl Triphenyl Phosphonium Chloride
Butyl Triphenyl Phosphonium Bromide
Butyl Triphenyl Phosphonium Chloride
Ethyl Triphenyl Phosphonium Acetate
Ethyl Triphenyl Phosphonium Bromide
Ethyl Triphenyl Phosphonium Iodide
Methyl Triphenyl Phosphonium Bromide
Tetrabutyl Phosphonium Bromide
Tetraphenyl Phosphonium Bromide
top of page
   Polyglycols and Crown Ethers
18—crown-6
Aliplex DB186
Butyl Diglyme
Dibenzo-18-crown-6
Diethylene Glycol Dibutyl Ether
Diethylene Glycol Dimethyl Ether
Diglyme
Dipropylene Glycol Dimethyl Ether
Monoglyme
Polyethylene Glycol Dibutyl Ether
Polyglycol BB 300
Polyglycol DME 200
Polyglycol DME 250
Polyglycol DME 500
Polyglycol DME 1000
Polyglycol DME 2000
Monoethylene Glycol Dimethyl Ether
Tetraethylene Glycol Dimethyl Ether
Tetraglyme
Triethylene Glycol Dimethyl Ether
Triglyme

1. Method of whitening teeth, comprising:
a) providing a whitening compound;
b) providing a phase transfer catalyst, the phase transfer catalyst being selected for enhancing the transfer of the whitening compound to a stain on a patient's tooth;
c) applying the whitening compound to a surface of the patient's tooth.

2. Method as in claim 1, wherein:
a) the phase transfer catalyst is provided on the patient's tooth prior to the application of the whitening compound.

3. Method as in claim 2, wherein:
a) the whitening compound includes an oxygen free radical.

4. Method as in claim 3, wherein:
a) the phase transfer catalyst includes at least one of a quaternary phosphonium salt, a polyglycol, and a crown ester.

5. Method as in claim 3, wherein:
a) the phase transfer catalyst includes a resin.

6. Method as in claim 2, wherein:
a) the phase transfer catalyst is provided as a rinse.

7. Method as in claim 1, wherein:
a) the whitening compound includes an oxygen free radical.

8. Method as in claim 1, wherein:
a) the phase transfer catalyst includes at least one of a quaternary phosphonium salt, a polyglycol, and a crown ester.

9. Method as in claim 1, wherein:
a) the phase transfer catalyst includes a resin.

10. Method as in claim 1, wherein:
a) the phase transfer catalyst is provided as a rinse.
